(19) United States
(12) Patent Application Publication
(10) Pub. No.: US 2023/0213503 A1
(43) Pub. Date: Jul. 6, 2023

(54) POINT-OF-CARE IMMUNOASSAY DEVICE AND METHOD

(71) Applicant: IgGENE Limited, Fo Tan (HK)

(72) Inventors: Pak Leong Lim, Ma On Shan (HK); Chi Hang Frankie Tam, Shatin (HK)

(21) Appl. No.: 18/149,707

(22) Filed: Jan. 4, 2023

(30) Foreign Application Priority Data
Jan. 4, 2022 (AU) 2022200014

(51) Int. Cl. G01N 33/533; G01N 33/577; G01N 33/543; B01L 3/00

(52) U.S. Cl. CPC G01N 33/533; G01N 33/577; G01N 33/54326; B01L 3/502715; B01L 3/50273; B01L 3/52; B01L 2400/0475; B01L 2200/10; B01L 2300/0867; B01L 2200/16; B01L 2300/0609; B01L 2400/043; B01L 2300/0663; B01L 2400/082

(57) ABSTRACT

An immunoassay device for use in quantitatively measuring an amount of an analyte in a fluid sample, employs reagents that include particle pairs comprising a) one of an antigen and antibody coupled with a label, and b) a magnetic particle coupled with the other of the antigen and antibody. A transport which moves a set of reaction wells along a path and a dispenser dispenses respective ones of the reagents into the reaction wells. Prior to magnetic separation and optical analysis, a controller that coordinates movement of the transport with operation of the pipette modules operates the transport to reciprocate the set of reaction wells along the path for mixing the fluid sample with the reagents.

### TECHNICAL FIELD

The present invention generally relates to a device and method for rapid automated quantitative determination of analytes in liquid samples by immunoassays at a Point Of Care (POC), particularly assays incorporating labels and magnetic particles.

### BACKGROUND OF THE INVENTION

Immunodiagnostics are increasingly used to detect many types of diseases and health conditions ranging from cancer and heart-attack to infections such as Covid-19. There are basically two types. One relies on the use of heavy (non-transportable) equipment such as auto-analyzers which provide accurate results with high through-puts but long turnaround times, typically of more than one hour. (See Euroimmun: https://www.euroimmun.com/products/automation/chlia/; Abbott Labs, https://www.corelaboratory.abbott/int/en/offerings/brands/architect). It should be noted that there is traditionally no provision in these auto-analyzers for vigorously and thoroughly mixing the reactants during the reaction process because ample time is allowed for the incubation and the reactants are usually non-particulate in nature. However, there are instances where very brief mixing is effected e.g., by centrifuging the reaction mixture and then bringing the motion to a sudden halt.

The other type comprises simple rapid tests, typically of less than 30 minutes, that can be manually performed at POC settings, but which provide results that are generally less accurate. In these tests, the results are often read by eye although recently, small image sensors or fluorescence readers (not auto-analyzers that actually perform tests) have been employed to improve the scoring of results such as those based on the lateral-flow technique. (See Quidel, https://www.quidel.com/immunoassays/sofia-tests-kits; Creative Diagnostics, https://www.creative-diagnostics.com)

In most immunodiagnostics, the antibody or antigen employed as the reagent to detect properties of the analyte of interest is used in its free naked form, or conjugated with a label, or immobilized on the surface of a reaction well (tube). Recently, however, several systems have used magnetic microspheres in suspensions to immobilize either the antibody or the antigen and used as suspensions. These particles have a magnetic core and a polymer shell with a functionally modified surface. The advantage here is the ease by which the bound reagent can be separated from the unbound reagent in solution through the use of magnetic force. The use of such particles in immunoassays, however, which dates back to the pioneering work done by the applicant (see Lim P L, Ko K H, Choy W F. 1989. J. Immunol. Methods 117:267-273. https://doi.org/10.1016/0022-1759(89)90149-X) is still relatively uncommon.

It is even less common to use another microsphere besides the magnetic particle to immobilize the antibody or antigen reagent. Such a two-particle system is used in the TUBEX® test developed by the applicant (Lim P L, Tam F C H, Cheong Y M, Jegathesan M. 1998. J. Clin, Microbiol. 36:2271-8. DOI: 10.1128/JCM.36.8.2271-2278.1998; Yan M Y, Tam F C H, Kan B, Lim P L. 2011. PLOS ONE 6:e24743. https://doi.org/10.1371/journal.pone.0024743). In this rapid POC test, the magnetic particle is coupled with an antigen, while the second microsphere, which also has a polymer shell but lacks a magnetic core, is coloured to serve as the reaction indicator and is coupled with the corresponding antibody. Both particles are used in liquid suspensions. The two particles will bind to each other specifically and rapidly when mixed together, and both will settle to the bottom of the well when magnetic force is applied. However, if the relevant antigen or antibody is present in a sample to be analyzed, this will block the interaction between the pair of microspheres and cause the indicator microspheres to remain suspended in solution, thus giving a positive score. The results are visually read based on the colour intensity (semi-quantitative). The assay format is as such based on inhibition unlike the direct-binding or capture (sandwich) format used by the majority of immunodiagnostic systems.

Vigorous multi-directional mixing of the reactants during the whole incubation period is a crucial feature of the TUBEX® test, and distinguishes it from most other systems, such as ones based on the lateral-flow technique or ELISA. Mixing not only enhances the chances of collision between the two microspheres to speed up the interaction, but also ensures that the binding between the two particles, as well as between each particle and the analyte, is real (specific) and not spurious (equivalent to the effect achieved by traditional washing). This type of mixing is different from the brief mixing of reactants done by pipetting or vortexing that is sometimes used in some diagnostic systems when the reagents are first introduced. This is also different from the mono-directional light rinsing (washing) of reactants due to the capillary flow of fluid in the lateral-flow technique.

Achieving thorough mixing in a small container is a challenge. In TUBEX®, this is done manually by first sealing the mouth of the V-shaped multi-channel reaction wells with tape (to prevent leakage of contents) and laying the set of reaction wells flat on their face (to provide a large surface for mixing), and then shaking the wells vigorously in a forward-and-backward motion for several min.

It is apparent that the manual TUBEX® test, although simple and fairly accurate, has several shortcomings. One is the mixing step, since this is cumbersome and can be incorrectly performed by some users. Another is the subjectivity and difficulty associated with reading of the colorimetric results by eye.

Thus, there exists a need for a device and method for performing rapid automated quantitative analyses of an analyte in a fluid sample which provides a more homogeneous analyte and reagent mix in the sample for enhancing the accuracy of the analysis. It is particularly desirable to provide a device that is small and portable or transportable for use in a POC rapid test but which offers the high-precision of an auto-analyzer.

Disclosed herein is a device that can autonomously perform the TUBEX® test without the inherent problems of the manual test but with much enhanced sensitivity. Critically, a way was found to mix the TUBEX® reagents effectively without the need for capping the reaction well nor laying it flat i.e. while the reaction well is stood upright, open-mouthed. Sensitivity was improved by changes made to the TUBEX® reagents and assay method, and the provision of a photosensitive detector in the device which can read the two-colour fluorescence of the new TUBEX® reagents.

TABLE 1
Determination of the best conditions for mixing
TUBEX ® reagents using stirrers

| Experiment # | 1 | 2 | 3 | 4 | 5 | | 6 | | 7 | 8 |
|---|---|---|---|---|---|---|---|---|---|---|
| Stirring speed | 200 | 200 | 200 | 200 | 200 | | 100 | | 250 | 200 |
| (rpm) | | | | | | | | | | |
| Angle of | 180 | 180 | 1440 | 180 | 180 | | 180 | | 180 | 180 |
| displacement (°) | | | | | | | | | | |
| Stirring time (min) | 2 | 4 | 4 | 6 | 6 | | 6 | | 4 | 8 |
| Stirrer type | F | F | F | F | P | F | P | P | P | P |
| (F = fin; P = | | | | | | | | | | |
| propeller; see | | | | | | | | | | |
| FIG. 11) | | | | | | | | | | |
| TUBEX score | 4 | 4 | 4 | 2 | 0 | 2 | 0 | 2 | 2 | 0 |
| (0 = best mixing; | | | | | | | | | | |
| 10 = worst) | | | | | | | | | | |

METHOD: Antibody-coupled blue-coloured microspheres (90 μl) were mixed with antigen-coupled magnetic particles (90 μl) using a rotary stirrer in Type A reaction well (FIG. 10a) at the specified conditions, then stood on a magnet and the colour of the supernatant read visually. Other details as in Yan MY, Tam FCH, Kan B, Lim PL. 2011. PLOS ONE 6:e24743.
https://doi.org/10.1371/journal.pone.0024743

TABLE 2
Determination of the best conditions for mixing TUBEX ®
reagents using a horizontally-reciprocating stage

| Experiment # | 1 | 2 | 3 | 4 | 5 |
|---|---|---|---|---|---|
| Rocking speed (mm/s) | 100 | 100 | 150 | 150 | 150 |
| Displacement of platform (mm) | 10 | 5 | 20 | 5 | 5 |
| Mixing time (min) | 4 | 4 | 4 | 4 | 3 |
| TUBEX score | 1 | 1 | 3 | 0-1 | 1 |
| (0 = best mixing; 10 = worst) | | | | | |

METHOD: Antibody-coupled blue-coloured microspheres
(87.5 μl) were mixed with antigen-coupled magnetic particles
(87.5 μl) in Type B reaction well (FIG. 10b) at the specified
conditions, then stood on a magnet and the colour of the
supernatant read visually. Other details as in Table 1.

TABLE 3
Determination of the best reaction well for mixing TUBEX ®
reagents using a horizontally-reciprocating stage

| | TUBEX score | | |
|---|---|---|---|
| | (0 = best mixing; 10 = worst) | | |
| Total volume of reaction mix | 150 μl | 175 μl | 200 μl |
| Type B reaction well | 1 | 1 | 2 |
| (rectangle—FIG. 10b) | | | |
| Type C reaction well | 1 | 1 | 1 |
| (parallelogram—FIG. 10c) | | | |
| Type D reaction well | 0 | 0 | 0 |
| (trapezium—FIG. 1) | | | |

METHOD: Different volumes of antibody-coupled blue-coloured
microspheres were mixed with different volumes of antigen-
coupled magnetic particles in different types of reaction wells
for 4 min on a reciprocating stage(150 mm/s, 5 mm
displacement distance), then stood on a magnet and the colour
of the supernatant read visually. Other details as in Table 1.

Table 1 shows the results of an experiment aimed at finding out whether tiny mechanical stirrers (see FIG. 11) could be used to mix a small volume of TUBEX® reagents in a reaction well while being stood upright. The results show that this method is not very efficient since high stirring speeds and long stirring times (>6 min) are required.

Table 2 shows the results of an experiment aimed at finding out whether a rocking platform could be used to mix a small volume of TUBEX® reagents in a reaction well while standing the reaction well upright on the platform. The results show the great potential of this method at the conditions used.

Table 3 shows the results of an experiment aimed at extending the experiment of Table 2 to find a design of the reaction well (see FIGS. 10a-10c) that is most conducive for mixing. The results show that the reaction well which has the cross-section of a trapezium is the best at the chosen conditions.

In a further study using the original V-shaped reaction wells in an upright position, mixing was done by manually pipetting the reaction mixture up and down repeatedly for up to 2 min, but only <90% completion could be achieved (data not shown).

### DISCLOSURE OF THE INVENTION

According to one aspect of the present invention there is provided an immunoassay device for use in performing rapid immunodiagnostic tests to quantitatively measure an amount of an analyte in a fluid sample, comprising:

a set of reaction wells;

a transport which moves the set of reaction wells along a path;

first and second reagent holders disposed alongside the path for holding respective reagents;

a dispenser configured for withdrawing reagent from the reagent holders and dispensing the reagent into ones of the reaction wells, wherein the reagents comprise: a labelled reagent including one of a binding pair coupled with a label, and a magnetic reagent including a magnetic particle coupled with the other of the binding pair;

a magnet disposed alongside the path for applying a magnetic field in a magnetization direction to the contents of the set of reaction wells such that bound pairs are thereby separated;

a photosensitive detector configured to quantitatively measure the amount of analyte; and

a controller operable to coordinate movement of the transport with operation of the dispenser for dispensing of the reagents and operating the transport to reciprocate the set of reaction wells along the path for mixing the fluid sample with the reagents.

Advantageously, the resulting mixing performance substantially contributes to a more homogeneous analyte and regent mix in the sample, while allowing for the relatively simple configuration of the machine. In addition to the controller operating the transport to reciprocate the set of reaction wells along the path for mixing the fluid sample with the reagents, the controller may operate the transport to reciprocate the set of reaction wells along the path after dispensing one of the reagents and before dispensing the other of the reagents for mixing the fluid sample with the one of the reagents. In addition, following mixing of the fluid sample and reagents, the results may be resolved by operating the magnet to separate the bound pairs of reagents from the unbound label. Thus, the device can autonomously perform the TUBEX test, and it avoids the need to make a colour determination manually, and offers greater sensitivity by the use of a photosensitive detector.

Preferably the label comprises one of a fluorescent label, a chemilumiscent label and a dye. Preferably the labelled reagent further comprises a non-magnetic particle, the non-magnetic particle coupled to the label and to the one of antigen and antibody.

Preferably the set of reaction wells comprises like reaction wells arrayed in a longitudinal direction, each well extending down from an opening to a closed end, each well having substantially the same cross section throughout its height.

Preferably the reaction wells are generally trapezium-shaped in cross section, with a pair of transversely opposing outer walls forming bases of the trapezium, the transversely opposing outer walls comprising at least opposing windows of transparent material, the outer walls aligned substantially parallel to the path.

Preferably the trapezium is an acute trapezium, the opposing walls are aligned in the longitudinal direction of the array and the reaction wells of the set are integrally formed.

Preferably the openings are arrayed in a top flange that is generally flat and elongated in the longitudinal direction and serves to integrally connect tops of the reaction wells.

Preferably the path is linear and parallel to the longitudinal direction.

Preferably the dispenser comprises first and second pipette modules, each pipette module dispensing reagent from a respective one of the reagent holders. Alternatively, the dispenser may comprise a single robotic arm that holds a different dispensing device to dispense each reagent.

Preferably the controller operates each pipette module to alternately draw in and expel the fluid sample and reagent for further mixing of the fluid sample with each reagent.

Preferably the controller operates each pipette module to draw in a first volume and subsequently dispenses a fraction of the first volume into each of the reaction wells. By not using the pipette module to mix the reagent and each sample, no pipette washing step is required.

Preferably the controller operates each pipette module to alternately draw in and expel one or each of the reagents for mixing the reagent prior to dispensing the reagent.

Preferably the device further comprises opposing jaws mounted on the transport, resilient means for urging one of the jaws toward the other from a released position to an engaged position in which the set of reaction wells is clamped between the jaws.

Preferably the jaws are elongated in the longitudinal direction and clampingly engage at least one of the outer walls, at least one of the jaws having a respective array of windows, such that each window can be disposed in registration with one of the outer walls of each reaction well.

Preferably the transport is moveable along the path under control of the controller to a release station, wherein the release station comprises at least one actuator that is moveable under control of the controller to abut and move the at least one of the jaws from its engaged position to its released position.

Preferably a pair of parallel linear guides on the transport support longitudinally opposing ends of the one of the jaws for transverse movement and the at least one actuator comprises a corresponding pair of actuators, each actuator moveable simultaneously under control of the controller to abut and move the at least one of the jaws from its engaged position to its released position.

Preferably each actuator comprises a shaft mounted in a linear bushing for movement between a retracted position and an extended position for abutting the one of the jaws, each actuator driven by a rotary motor that turns a cam, wherein a cam follower engaged with the cam is connected to the shaft such that a lobe of the cam displaces the cam follower and the shaft to the extended position.

In another aspect, the invention provides an immunoassay method for quantitatively measuring an amount of a first analyte in a fluid sample, comprising:

providing a transport that moves along a path;

providing a set of reaction wells holding a fluid sample mounting the set of reaction wells to the transport;

providing in respective ones of two reagent holders a) a labelled reagent including one of a binding pair coupled with a label, and b) a magnetic reagent including a magnetic particle coupled with the other of the binding pair;

operating a dispenser for withdrawing reagent from one of two reagent holders;

coordinating movement of the transport with operation of the dispenser to dispense the withdrawn reagents into one of the reaction wells, and,

operating the transport to reciprocate the set of reaction wells along the path for mixing the fluid sample with the reagents,

before subsequently applying a magnetic field in a magnetization direction to the contents of the set of reaction wells such that bound are thereby separated, and

operating a photosensitive detector to quantitatively determine the amount of the first analyte.

Preferably the method is performed using the immunoassay device described above, wherein one of the magnetic reagent and labelled reagent is dispensed into the reaction wells by the first dispenser of the immunoassay device and other of the magnetic reagent and labelled reagent is dispensed into the reaction wells by the second dispenser of the immunoassay device.

Alternatively the method is performed using the immunoassay device described above, wherein one of the magnetic reagent and labelled reagent is dispensed into the reaction wells by the dispenser of the immunoassay device and other of the magnetic reagent and labelled reagent is dispensed into the reaction wells outside of the immunoassay device.

Preferably the method comprises first and second mixing periods during which the transport is operated to reciprocate the set of reaction wells along the path for mixing, the first mixing period following the dispensing of one of the magnetic reagent and labelled reagent, the second mixing period following the dispensing of the other of the magnetic reagent and labelled reagent.

Preferably the label comprises one of a fluorescent label, a chemilumiscent label and a dye.

Preferably the labelled reagent further comprises a first particle, the first particle coupled to the label and to the one of the binding pair.

Preferably the method further comprises adding to the fluid sample a second reagent pair, the second reagent pair having a specificity and label differing from those of the first reagent pair, and

operating the photosensitive detector to quantitatively determine the amount of a second analyte.

In this manner advantage is also taken of the availability of many different types of fluorophore with distinct excitation and emission frequencies to mix two or more fluorophores together in a single test, so that multiple specificities can be simultaneously examined.

Preferably, for the detection of an antigen, the binding pair comprises an antigen and antibody pair and the first particle is coupled with the antibody, and the second particle is coupled with the antigen, and the transport first reciprocates the reaction wells to mix the analyte and one of the reagents of the first reagent pair, before dispensing of the other of the reagents of the first reagent pair.

Preferably, for the detection of an antibody, the binding pair comprises an antigen and antibody pair and the first particle is coupled with the antigen, and the magnetic particle is coupled with the antibody, and the transport first reciprocates the reaction wells to mix the analyte and one of the reagents of the first reagent pair, before dispensing the other of the reagents of the first reagent pair.

Preferably the photosensitive detector is used to measure one of florescence, chemiluminescence and colour.

Preferably the results derived from the photosensitive detector are output in analog form, with an indicator highlighting where a result lies on a scale.

The method may further comprise adding to the fluid sample a second reagent pair, the second reagent pair having a specificity and label differing from those of the first reagent pair, and

operating the photosensitive detector to quantitatively determine the amount of a second analyte.

Preferably the antibody used for coupling the first particle is a mixture of monoclonal antibodies.

Preferably the antibody used for coupling the magnetic particle is a mixture of monoclonal antibodies.

Preferably the dispenser comprises first and second pipette modules, and wherein operating the dispenser comprises withdrawing reagent from a respective one of the two reagent holders using a respective one of the first and second pipette modules.

In still another aspect, the invention provides an immunoassay method for quantitatively measuring an amount of a first analyte in a fluid sample, the sample further comprising a first reagent pair comprising a) a labelled reagent including one of a binding pair coupled with a label, and b) a magnetic reagent including a magnetic particle coupled with the other of the binding pair, the method comprising:

providing a transport that moves along a path;

providing a set of reaction wells holding a fluid sample

mounting the set of reaction wells to the transport;

operating the transport to reciprocate the set of reaction wells along the path for mixing the fluid sample with the reagents,

before subsequently applying a magnetic field in a magnetization direction to the contents of the set of reaction wells such that bound are thereby separated, and

operating a photosensitive detector to quantitatively determine the amount of the first analyte.

Preferably the reaction wells are generally trapezium-shaped in cross section, with a pair of transversely opposing outer walls forming bases of the trapezium, the transversely opposing outer walls comprising at least opposing windows of transparent material, the outer walls aligned substantially parallel to the path.

### BRIEF DESCRIPTION OF THE DRAWINGS

Preferred forms of the present invention will now be described by way of example with reference to the accompanying drawings, wherein:

FIG. 1 is a pictorial view from above showing a set of reaction wells of the immunoassay device of the invention;

FIG. 1a is a schematic longitudinal section through the set of reaction wells of FIG. 1;

FIG. 2 is a pictorial view of a first embodiment of the immunoassay device of the invention;

FIG. 3 is a pictorial view of a pipette module of the immunoassay device of FIG. 2;

FIG. 4 is a pictorial view of a reagent holder of the immunoassay device of FIG. 2;

FIG. 5 is a pictorial view of a subassembly of the immunoassay device of FIG. 2 that includes a transport and two actuators;

FIG. 6 is an end elevation of the subassembly of FIG. 5;

FIG. 7 is a pictorial view of the transport of the subassembly of FIG. 5;

FIG. 8 is a pictorial view of one of the actuators of the subassembly of FIG. 5;

FIG. 9 is a schematic of the fluorescence detector of the immunoassay device of FIG. 2.

FIGS. 10a, 10b and 10c each contain three side views, a plan view and an oblique view of respective different reaction wells used in the experiments described in the background to the invention;

FIGS. 11a and 11b each contain a set of views of a propeller-type stirrer and fin-type stirrer, respectively, used in the experiments to mix the TUBEX® reagents described in the background to the invention;

FIGS. 12a and 12b are graphs showing the sensitivity of the TUBEX® test performed according to the invention using fluorescent indicator particles and comparing it with that of the manual TUBEX® test using coloured indicator particles for both the detection of antibody (A) and antigen (B) in the fluid sample;

FIG. 13 is a fragmentary pictorial view of a second embodiment of the immunoassay device of the invention;

FIG. 14 is a pictorial view of the transport and well holder of FIG. 13;

FIG. 15 is a pictorial view of the fluorescence detector of FIG. 13;

FIG. 16 is a pictorial view of magnet mount of FIG. 13;

FIG. 17 is a fragmentary end elevation showing the reaction well set and adjacent components of the fluorescence detector of FIG. 13.

### DESCRIPTION OF THE PREFERRED EMBODIMENTS

As used herein, the terms “antibody” and “antibodies” refer to serum proteins classified as immunoglobulins (Ig); and includes (a) the various isotypes such as IgM and IgG, (b) intact whole molecules or fragments such as single-chain Fv or camelids, (c) both natural and re-engineered forms, and (d) both monoclonal and polyclonal sources including mixtures of monoclonal antibodies.

A ‘surrogate antibody’ can substitute for an antibody, and means any substance with a structure different from that of an antibody, that is natural or chemically-synthesized, and that has a suitable binding affinity for the antigen. Surrogate antibodies can come human, viral and plant sources. For instance, a suitable ligand for the Covid-19 spike protein is the angiotensin-converting enzyme receptor protein (ACE2); another is the various plant lectins that bind to various glycoproteins.

‘Antigen’ refers to any substance containing one or more antigenic sites (epitopes), natural or chemically-synthesized, intact or fragmented, that can be bound by an antibody through the epitopes; the size can range from small chemical groups with a single epitope such as tyvelose, to serum proteins or microbial extracts with multiple epitopes, and to even larger entities such as whole microorganisms, viruses, and blood cells. Sub-specificities refers to the different epitopes present in an antigen that are recognized by individual monoclonal antibodies.

A ‘binding pair’, includes a ‘complementary binding pair’, and comprises an antigen and antibody pair, and an antigen and surrogate antibody pair.

‘Microsphere’, ‘bead’ or ‘particle’ refers to particulate matter composed of polystyrene or silica with a diametric size ranging from 10 nm to 10 μm. Particles are coated or coupled with an antigen or antibody by covalent or non-covalent means, directly or indirectly via spacers or adaptors such as protein G, using conventional methods known to those skilled in the art.

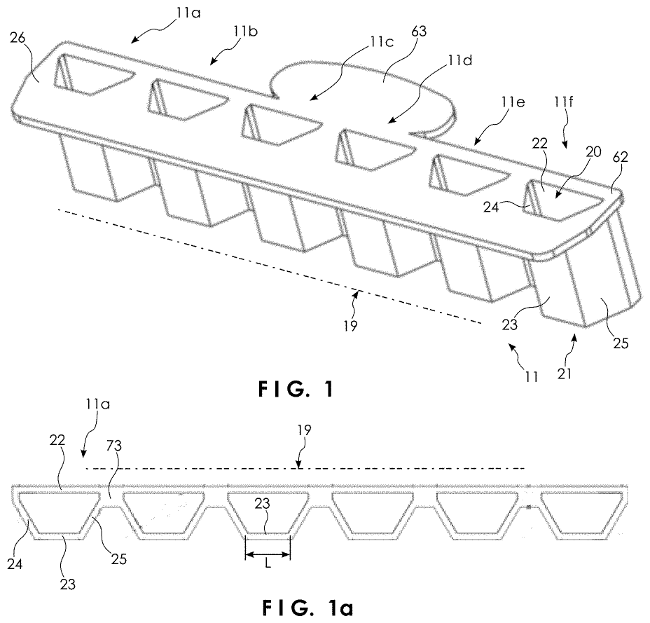

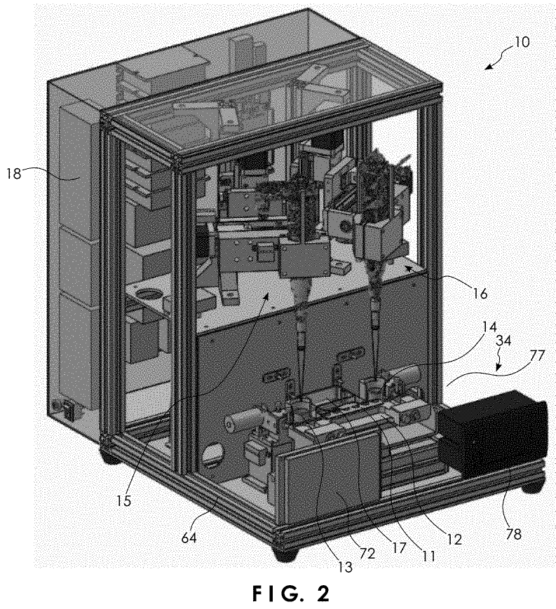

Referring particularly to FIGS. 1 and 2, the first embodiment of the immunoassay device 10 of the invention is a self-contained benchtop auto-analyser that is portable and may be battery powered, or mains powered. The functional units of the device 10 include a set of reaction wells 11, a transport 12, first and second reagent holders 13, 14, a dispenser 15, 16 (comprising first and second pipette modules 15, 16), electromagnets 38, a fluorescence detector 17 and a controller 18. The reaction between the reagents and the test sample is performed in the set of reaction wells 11 which are automatically delivered in sequence from the first pipette module 15 to the second pipette module 16 along a path 34, which extends linearly to move the set of reaction wells 11 on to the fluorescence detector 17 to complete the assay.

As best seen in FIGS. 1 and 1a, the set of reaction wells 11 comprises like reaction wells 11a-11f arrayed along a longitudinal axis 19. Each well 11a-11f extends down from an opening 20 to a flat closed end 21, and each may have substantially the same generally trapezium-shaped cross section throughout its height. A pair of transversely opposing outer walls 22, 23 of each well may be planar and parallel. These outer walls 22, 23 form bases of the generally trapezium-shaped cross section and are longitudinally aligned, as with the long outer walls 22 being substantially coplanar with one another and the short outer walls 23 being substantially coplanar with one another. At least these opposing walls 22, 23 are formed of transparent material, or include transversely aligned windows of transparent material. Most preferably, the entire set of reaction wells 11 are integrally formed in one piece of transparent polycarbonate or glass. While the walls 22, 23 are most preferably planar and parallel, they might, for instance be curved, as to be concave or convex in horizontal cross section (although this may require optical compensation in the fluorescence detector 17).

Transverse walls 24, 25 of each well are also flat and join the outer walls 22, 23 forming legs of the generally trapezium-shaped cross section that are acutely inclined to the longitudinal axis 19 such that the trapezium is an acute trapezium, particularly an isosceles trapezium. The outer walls 22, 23 and transverse walls 24, 25 may have the same thickness. In the generally trapezium-shaped cross section, the long wall 22 may have a length, in the direction of the longitudinal axis 19, of between 150-250% of the length L of the short wall 23, with the transverse spacing between the walls of between 80-120% of the length L. A top flange 26 that may be generally flat and elongated in the longitudinal axis 19 serves to integrally connect tops of the reaction wells 11a-11f, while a web 73 may extend vertically between the closed end 21 and the flange 26 to connect tapered ends of adjacent ones of the wells 11a-11f throughout their length. As shown in FIG. 1a, in a horizontal plane through the wells, the web 73 may have dimensions that exceed the thickness of the walls 22-25. The openings 20 may be arrayed in this top flange 26. At least one of the longitudinal edges of the top flange 26 may project transversely from the adjacent one of the outer walls 22, 23 to form a lip 62. A central section of the lip 62 may include a projecting tab part 63.

The above-described set of reaction wells 11 has been found to offer advantageous mixing performance that substantially contributes to a more homogeneous analyte and regent mix in the sample, while allowing for the relatively simple configuration of the machine. This is achieved by aligning the longitudinal axis 19 parallel to the linear path 34, and operating the transport 12 to reciprocate linearly along the path 34. It is believed, without wishing to be limited by theory, that, owing to inertial effects, a rotational component of movement is imparted to the fluid when it impacts the transverse walls 24, 25 when the wells are sharply decelerated at the opposing ends of its longitudinal movement. This rotational component is in opposite directions at the opposite ends of the longitudinal movement, and so reciprocating with a sufficiently high amplitude and suitable frequency, such as an amplitude greater than or equal to the longitudinal dimension of the long wall 22 at between 10 and 50 Hz, ensures a turbulent flow regime that promotes vigorous mixing. By ensuring the wells are between no more than about 20 or 30% full with liquid ensures that no loss or overflow through the openings 20 occurs, so it is unnecessary to provide a closure over the openings 20.

A pair of reagents for use in the device 10 may comprise a labelled reagent including antigen-bound fluorescently labelled microspheres and a magnetic reagent including magnetic microspheres bound with a corresponding antibody. For instance, for a Covid-19 assay to detect antigen from a nasal swab, the magnetic reagent may include magnetic microspheres coated with an antibody specific to the nucleocapsid (NP) of the Covid-19 virus, while the labelled reagent may include microspheres dyed with fluorescein of a certain colour and coated with the corresponding Covid-19 nucleocapsid antigen. When mixed together with the liquid sample, the fluorescein-labelled microspheres and magnetic microspheres bind to one another in the antigen-antibody reaction and, if Covid-19 antigen is absent from the sample, when the magnetic microspheres and unbound fluorescein-labelled microspheres are separated by the application of a magnetic field, no fluorescein-labelled microspheres will be left in suspension and the result will thus be negative. However, when Covid-19 antigen (or the corresponding antibodies) are present in the liquid sample, these antigen or antibodies will block the binding between the pairs of microspheres. Significant amounts of fluorescein-labelled microspheres will be left unbound and remain suspended after the magnetic microspheres are separated by the application of a magnetic field, the degree depending on the amount of inhibitor (antigen or antibodies) present in the sample. The fluorescence detector 17, by measuring fluorescent intensity provides quantitative measurement.

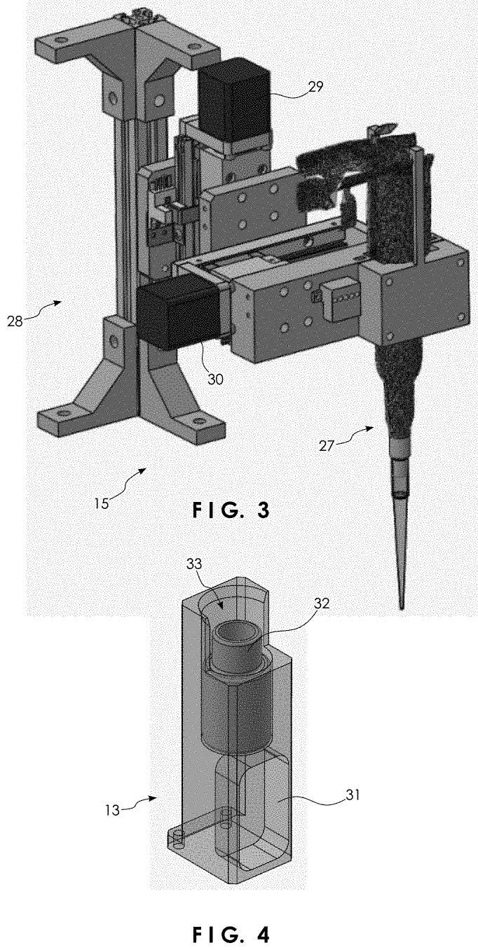

In a preferred embodiment, two reagent pairs, each pair with a different specificity and different fluorophore, are added to a single well. The emission of both is measured and different analyte concentrations are calculated from these two emission signals, allowing two tests to be performed simultaneously from the same sample. For instance, one of the pairs (of a first test) may include antibody-bound microspheres labelled by a fluorophore with an emission wavelength of 525 nm (and the corresponding antigen-bound magnetic particles), while the other of the pairs (of the second test) includes microspheres conjugated with an antibody of a different specificity and labelled by a fluorophore with a different emission wavelength e.g. 575 nm. The first pipette module 15 is shown separate from the device 10 in FIG. 3, and may have like construction to the second pipette modules 16, differing only in handedness. The pipette module 15 may comprise a motorised pipette 27 mounted to a robotic arm 28 that is carried on a frame 64 of the device 10. Movements of the robotic arm 28 and operation of the motorised pipette 27 to draw in and expel a reagent may be coordinated by the controller 18 to automatically fill the reaction wells 11a, 11b, 11c, 11d with respective reagents from the reagent holders 13, 14. The robotic arm 28 may include two linear degrees of freedom, particularly an upright translation axis powered by drive unit 29 and horizontal translation axis powered by drive unit 30. This provides for vertical translation of the pipette, for lowering the pipette 27 into both the reaction wells 11a, 11b, 11c, 11d and into the mouth of the reagent holder 13. The horizontal translation axis allows the motorised pipette 27 to be moved between the positions above the reagent holder 13 and above the linear path 34 upon which the reaction wells 11 are moved by the transport 12.

As shown in FIG. 4, the reagent holder 13 may have like construction to the reagent holder 14, each holding a respective one of the labelled reagent and magnetic reagent. The reagent holder 13 may comprise a mount 31 that may be fixed to a frame 64 of the device 10 with an upwardly-opening recess in which a container 32 is received. The container 32 has an upwardly facing mouth 33 for receiving the pipette 27. The controller 18 operates each pipette module 15, 16 to alternately draw in and expel each of the reagents for mixing prior to dispensing to ensure homogeneity.

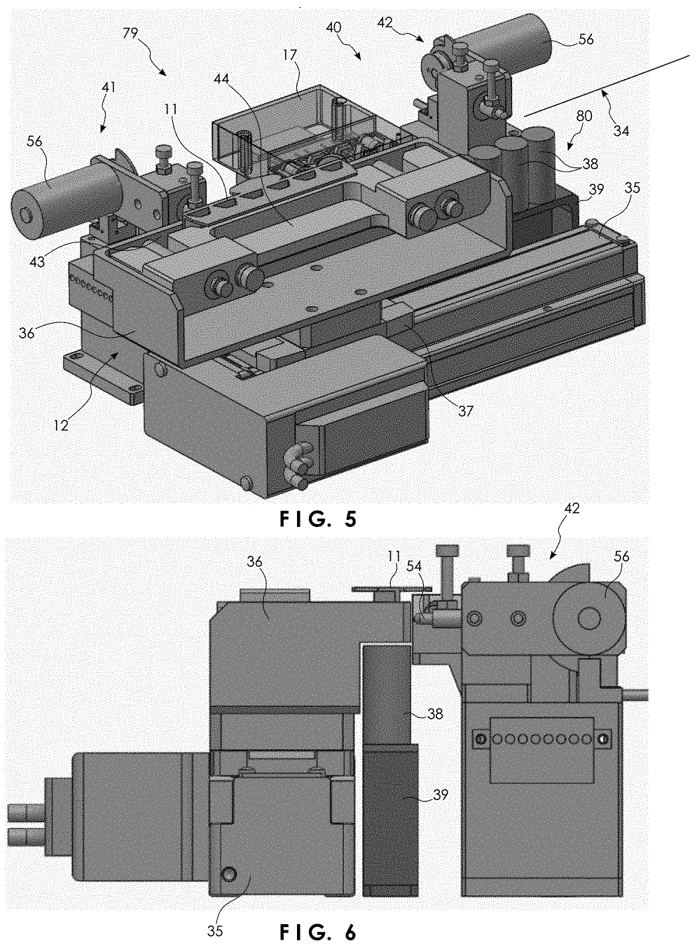

Referring to FIGS. 5 and 6, the transport 12 moves the set of reaction wells 11 along a path 34 defined by a guideway 35 that is horizontal, linear and parallel to the longitudinal axis 19. The transport 12 may be an assembly comprising a well holder 79 that holds the set of reaction wells 11 and is fixed to a linear bearing 37 that is mounted on the guideway 35 and which is driven as by a linear actuator such as a hydraulic ram (not shown) that extends longitudinally adjacent the guideway 35. By controlling the linear actuator, the controller 18 can thus move the transport 12 along the path 34 in either direction with a desired velocity profile, and also precisely locate the reaction wells 11a, 11b, 11c, 11d below the pipette 27, as needed. A magnet mount 80 mounts the electromagnets 38, and includes a pedestal 39 alongside the guideway 35 such that the closed ends 21 are positioned vertically spaced apart from, and overlying, the upper ends of the electromagnets 38 such that, when operated by the controller 18, a magnetic field pulls the magnetic microspheres toward the closed ends 21.

The transport 12 is moveable along the path 34 under control of the controller 18 to a release station 40, wherein longitudinally opposite ends of the transport 12 are disposed adjacent actuators 41, 42 and the set of reaction wells 11 is intermediate therebetween. Clamping the set of reaction wells 11 to the transport 12, the well holder 79 has a fixed jaw 43 which may be integral with the table 36 and which cooperates with an opposing moving jaw 44. A pair of parallel linear guides 45, 46 may be fixed to longitudinally opposing ends of the moving jaw 44 and received to slide in respective bushings 47, 48 mounted to the table 36 to support the moving jaw 44 for transverse movement. Springs 49, 50 are mounted about respective support bars 51, 52 aligned parallel to the linear guides 45, 46 and resiliently urge the moving jaw 44 toward the fixed jaw 43 and its engaged position. The jaws 43, 44 are elongated in the longitudinal axis 19 and have respective upright planar faces that abut and clampingly engage the set of reaction wells 11, with the moving jaw 44 abutting the outer walls 23 and the opposing fixed jaw 43 abutting the outer walls 22. Features (not shown) on one of the jaws 43, 44 may ensure that the set of reaction wells 11 can be accurately clamped in only one position and orientation. The fixed jaw 43 may include an array of windows 53, each disposed in registration with one of the outer walls 22 of each reaction well, and disposed opposite an aligned array of windows in the moving jaw 44.

With the transport 12 at the release station 40, the actuators 41, 42 are moveable simultaneously under control of the controller 18 to abut and move the moving jaw 44 from its engaged position to its released position. The actuators 41, 42 are of like construction, and differ only in handedness. Each actuator may comprise a shaft 54 mounted in a linear bushing 55 for movement between the retracted position shown and an extended position (not shown) in which it abuts the moving jaw 44. Each actuator 41, 42 may be driven by a rotary motor 56 that turns a cam 57, wherein a cam follower 58 engaged with the cam 57 is connected to the shaft 54 such that a lobe 59 of the cam 57 displaces the cam follower 58 and the shaft 54 to the extended position. A spring (not shown) may serve to retract the shaft 54 and urge the cam follower 58 into engagement with the cam 57. In the extended position, the shafts 54 pass through apertures 60, 61 in the fixed jaw 43 to abut the opposite ends of the moving jaw 44.

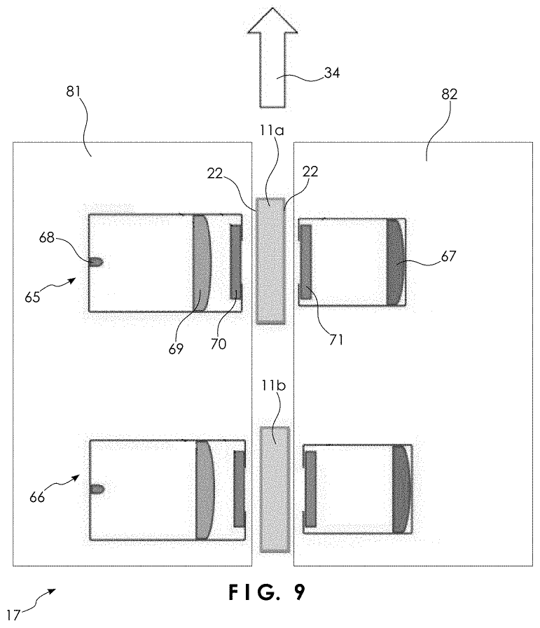

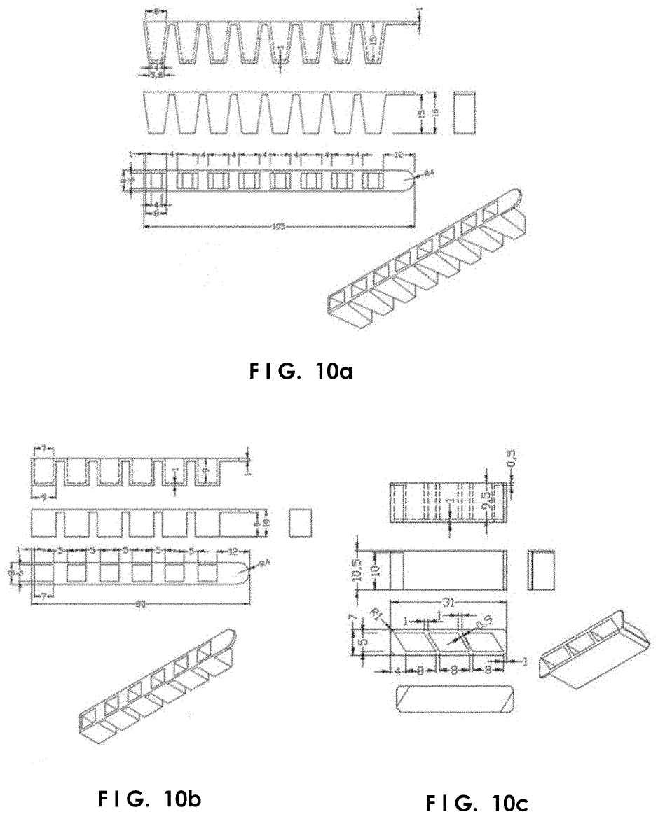

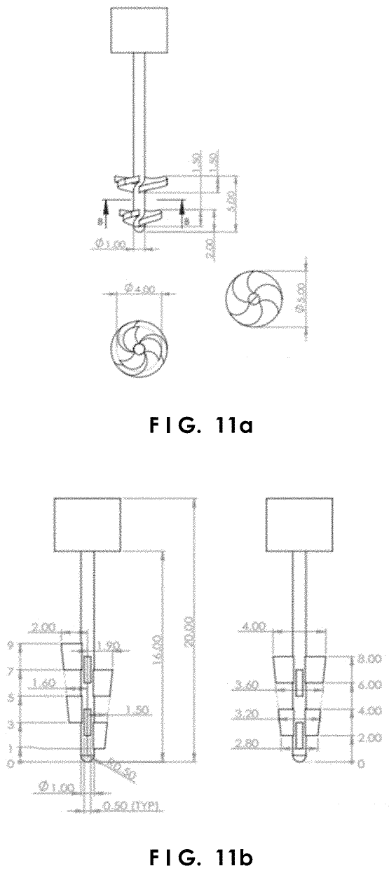

The fluorescence detector 17 may include two like reader instruments 65, 66 adjacent one another and spaced apart along the path 34, each with a respective optical channel orthogonal to the path 34. Each optical channel is also orthogonal to the plane defined by the outer walls 22 of the wells 11. In this manner, diffraction losses are mitigated and the transport 12 may be moved along the path 34 in a stepwise manner, to align each optical channel with one of the wells 11a, 11b, 11c, 11d etc successively to perform the fluoroscopy. The spacing between the reader instruments 65, 66 along the path 34 may equal the longitudinal spacing between adjacent ones of the wells, allowing adjacent wells to be read simultaneously. Each reader instruments 65, 66 may include excitation 70 and emission 71 filters, a light source 68, plano-convex lens 69 and an emissions sensor 67. The emissions sensors 67 generate an output voltage in response to fluorescence emissions excited by the light source 68. The light source 68 is preferably an LED with a dispersion angle of about 15°. These emissions sensors 67 may include photodiodes, photovoltaic devices, phototransistors, avalanche photodiodes, photoresistors, CMOS, CCD, CIDs (charge injection devices), photomultipliers, and reverse biased LEDs, for instance. By the use of two emissions sensors 67 the fluorescence detector 17 is thus adapted to perform the above-described two different measurements simultaneously. The pair of exciter parts of the instruments 65, 66 on one side of the path 34 may comprise an exciter subassembly 81, with the opposing pair of receiver parts comprising a separate receiver subassembly 82 (shown schematically by rectangles in FIG. 9).

The fluorescence signals are instantaneously processed and calibrated against a standard curve by an onboard chip and eventually displayed as digital (numerical) values. In addition, the results are also colour-coded to simplify interpretation for POC users. For example, ‘brown’ can be used to denote results that fall between the 0th and 10th percentile which are considered ‘negative’, ‘yellow’ as ‘borderline positive’ for values that lie between the 11th and 20th percentile, ‘green’ as ‘positive’ for values that lie between the 21st and 50th percentile, and finally, ‘blue’ as ‘strong positive’ for values over the 51st percentile.

Position sensors associated with moving parts of the device 10 provide feedback to the controller 18 to ensure each moving part is correctly configured at each stage of operation before moving to a subsequent stage, in the manner well known in the automation arts.

The device 10 may be operated with the labelled reagent and magnetic reagent in the reagent holders 13, 14. In use, with the moving jaw 44 released, and after placing a patient's fluid samples in the wells 11a, 11b, 11c, 11d, the operator may place the set of reaction wells 11 between the jaws 43, 44 where it may rest upon the table 36. In this position, the device 10 is ready to be started. Optionally, if the batch size is smaller than the number of wells not all of the wells will contain samples, and so the operator provides an initial input to the controller, as via a key pad (not shown) to identify the wells holding samples. After the operator provides a start command, the controller 18 operates the actuators 41, 42 to move together to their retracted positions, allowing the moving jaw 44 to move under the resilient action of the springs 49, 50 and firmly clamp the wells 11 between the jaws 43, 44. The controller 18 operates the first and second pipette modules 15, 16, controlling the robot arm to move a tip of the pipette 27 down into each container 32. To mix the reagents, a volume of reagent is repeatedly drawn in and expelled, before the pipette 27 draws in a predetermined amount sufficient to complete the batch. The controller 18 may then operate the transport 12 to place each well in turn in a first filling position on the path 34 adjacent the first pipette module 15. At this first filling position the pipette 27 is lowered into the well and a predefined volume of the labelled reagent of a first reagent pair is dispensed, before the pipette 27 is withdrawn, this operation of the first pipette module 15 being alternated with stepwise movement of the transport 12. After each well has received the labelled reagent of the first reagent pair in this manner, the pipette returns to the container 32 and ejects remaining reagent into the container. The controller 18 controls the transport 12 to reciprocate along the path 34, as at 30 Hz with an amplitude of 5 mm for two minutes, to mix the reagents and sample. The controller 18 operates the transport 12 to move each well in turn in a second filling position on the path 34 adjacent the second pipette module 16 and the corresponding filling steps are performed in the same manner to dispense the magnetic reagent of the first reagent pair into each of the wells. Next, the controller 18 controls the transport 12 to reciprocate along the path 34 in the above-described manner for a pre-defined period to mix the reagents and sample. A different labelled reagent and magnetic reagent, comprising a second reagent pair with a specificity different to that of the first reagent pair, are then dispensed into each well in a like manner.

The controller 18 operates the transport 12 to place the wells 11 over the electromagnets 38 which are then supplied with current to draw the magnetic microspheres and bound fluorescein-labelled microspheres down to the closed ends 21. After a predetermined time sufficient to complete the magnetic separation, the controller 18 operates the device 10 to perform the fluoroscopy. Readings are taken by the fluorescence detector 17 for each sample. The reader instrument 65 receives the first well 11a and the fluoroscopy is completed on the contents of this first well 11a, before the wells 11 are indexed forward one step so that reader instrument 65 receives the second well 11b and reader instrument 66 receives the first well 11a. In this position, and corresponding positions between the first and last, the reader instruments 65 and 66 are operated simultaneously. The reader instruments 65 and 66 have differing excitation 70 and emission 71 filters, and thus measure different emissions for different simultaneous tests.

By aligning, in turn, each well and corresponding window 53 with the reader instruments 65, 66 by stepwise displacement of the transport 12. The transport 12 may be maintained stationary, or else each scan may be performed following a like movement profile, while the fluorescence detector 17 is operated to produce the fluoroscopic reading for each sample. The controller 18 processes signals from the fluorescence detector 17 to quantitatively measure the amount of analyte, producing a reading for each sample analysed and which may be formatted by the controller 18 and sent to a display 72, to a printer 78 or, for instance, wirelessly to a connected computer.

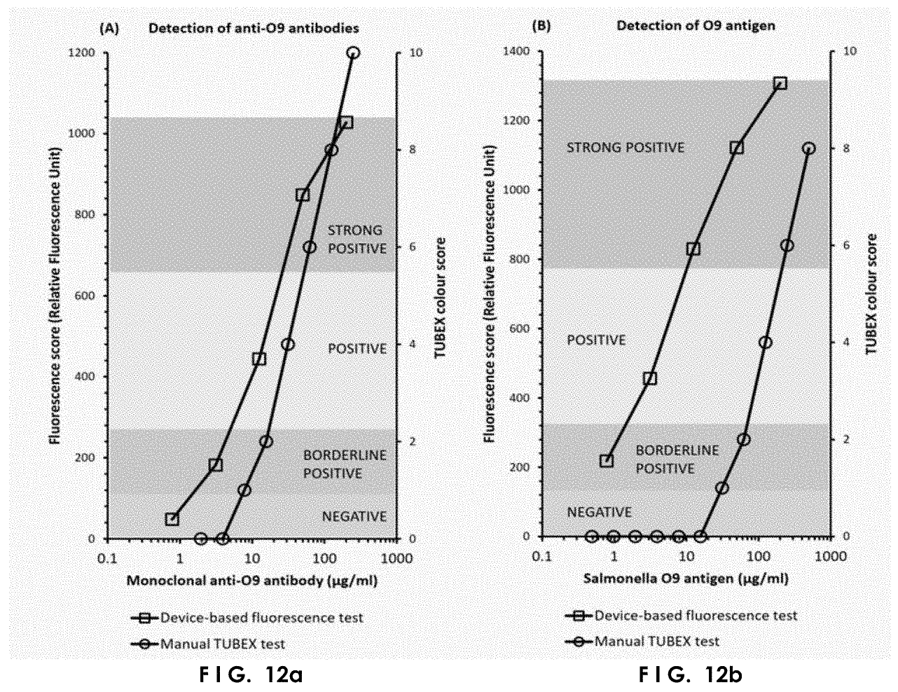

Referring to FIGS. 12a and 12b, these graphs show the sensitivity of the TUBEX test performed according to the invention using fluorescent indicator particles and comparing it with that of the manual TUBEX test using coloured indicator particles for both the detection of antibody (A) and antigen (B) in the fluid sample. Fluorescence was measured using a sensor (see FIG. 9) and expressed as numerical values (mV). In addition, the results are coloured differently according to their significance: ‘Negative’, ‘Borderline Positive’, “Positive’, and ‘Strong Positive’.

The same O9 antigen-coupled magnetic particles were used throughout and the same monoclonal anti-O9 antibody was coupled to both the coloured and fluorescent indicator particles (all particles purchased from Merck Co., Paris, France). It is apparent that the device (invention)-based results are superior, particularly in the case of antigen detection where the analyte was pre-mixed (2 min) with the indicator particles before mixing with the magnetic particles (4 min). Mixing was performed in trapezium-type reaction wells using the device (see Table 3) or manually in V-shaped reaction wells in a shaker. Details of the latter method including the reagent particles used are described in Yan M Y, Tam F C H, Kan B, Lim P L. 2011. PLOS ONE 6:e24743. https://doi.org/10.1371/journal.pone.0024743

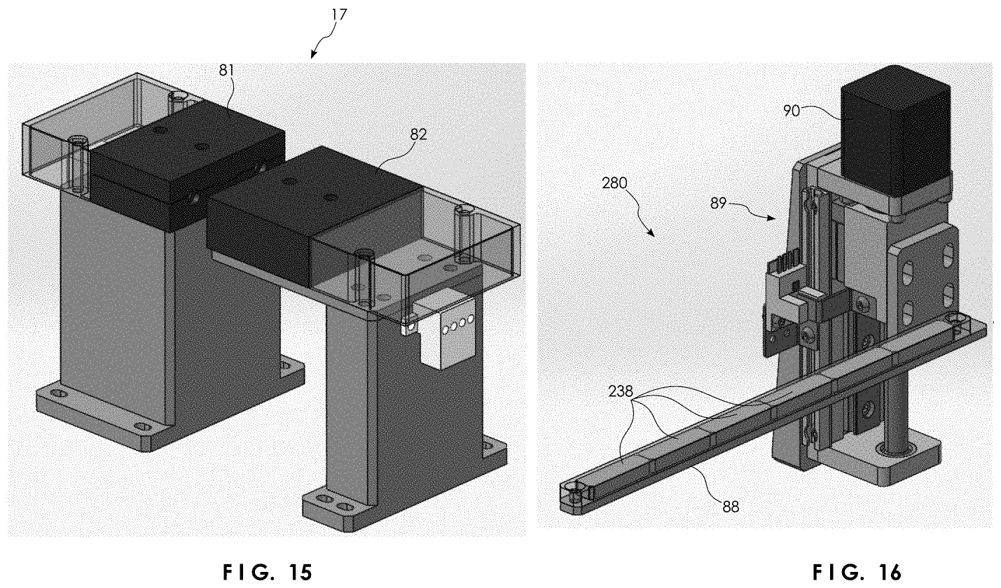

A second embodiment of the immunoassay device 210 is shown in FIGS. 13-17, and has the same configuration, and largely the same construction, as that of the first embodiment of the immunoassay device 10, but it includes an alternative construction for the well holder 279 and magnet mount 280. As a consequence, the device 210 operates differently to accommodate these, generally simplifying, changes. Beyond the well holder 279 and magnet mount 280, the parts are alike, and like reference numerals are used below to indicate like parts to those referenced in the first embodiment.

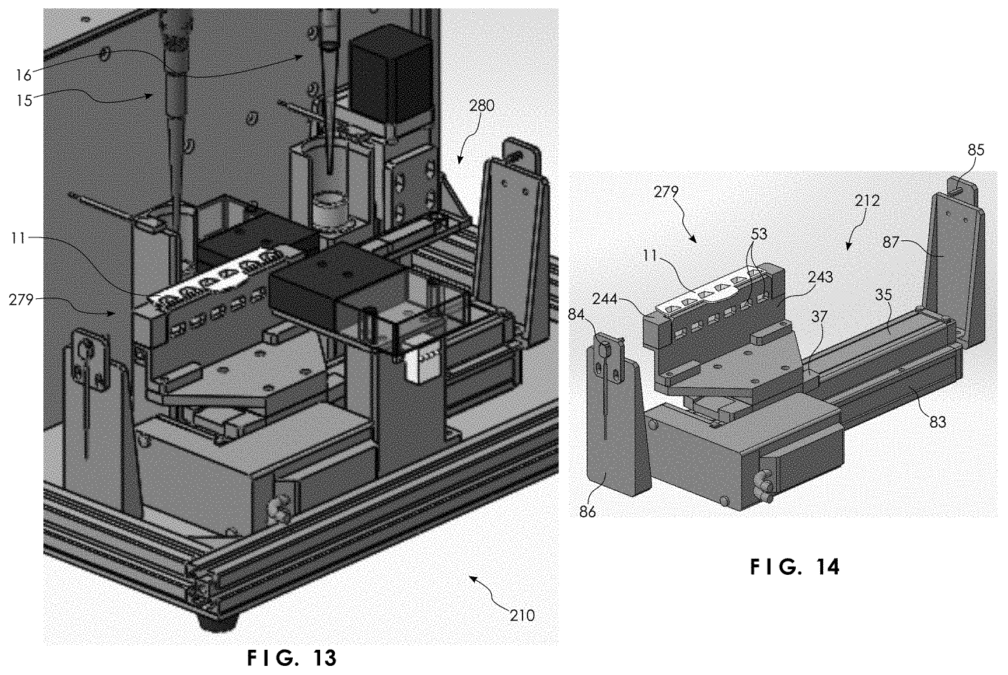

The well holder 279, best seen in FIG. 14, is part of a transport 212 moveable along the guideway 35 formed on the top of a bed 83. The guideway 35 cooperates with guides (not shown) on the well holder 279 to define the path 34 along which the set of wells 11 is reciprocated under control of the controller 18. In place of the sliding jaw 44, the well holder 279 includes a fixed second jaw 244 secured to the fixed jaw 243 to define an upwardly-opening recess complementary to the set of wells 11 and with an open top through which the set of wells 11 is inserted and withdrawn. Windows 253 in the second jaw 244 are disposed in registration with the windows 53 in the jaw 243. The tab 63 and flange 26 may project from opposite longitudinal edges of the jaws 243, 244. The fit between the set of wells 11 and the recess i.e. the jaws 243, 244 may be a friction fit or light interference fit, to avoid any relative movement therebetween during use, and means may be provided to make small position adjustments of the second jaw 244 relative to the first jaw 243 for setting this fit. Alternatively, complementary internal and external tapers on the recess and set of wells 11 respectively, narrowing toward the bottom of the recess, may provide for location and secure holding of the wells. FIG. 14 also shows limit switches 84, 85 which are position sensors connected to the controller and mounted on respective stanchions 86, 87 to contact the well holder 279 at the opposite ends of its linear travel.

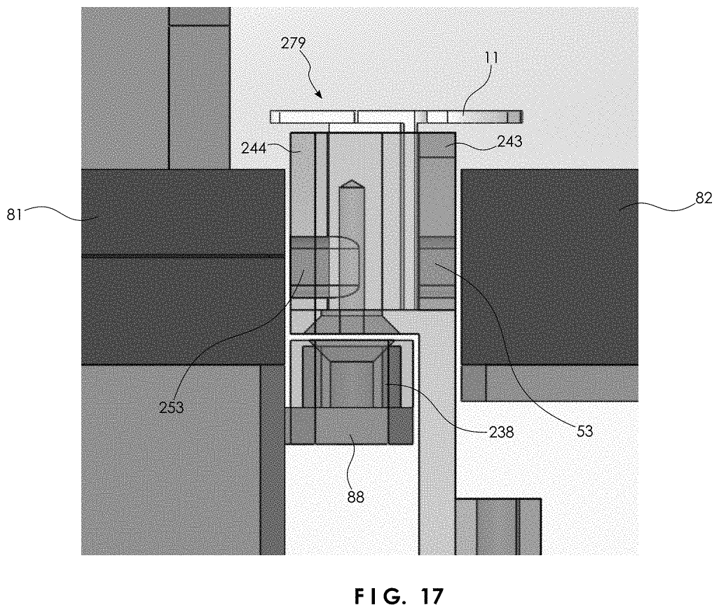

The magnet mount 280 mounts permanent magnets 238 in a linear array upon a beam 88 fixed in a cantilevered manner to project from an upright linear actuator 89 disposed alongside the guideway 35. The upright linear actuator 89 may be of the screw type, where the screw (not shown) is turned by a rotary electric motor 90. By adjusting the height of the permanent magnets 238, by a command from the controller 18, the magnetic field applied during operation to the contents of the set of wells 11 may be varied from zero, or a negligible level, up to the design level when raised to the position shown in FIG. 17, immediately below the set of wells 11.

In operation, the well holder 279, lacking motorised parts of the first embodiment, securing the set of wells 11 is simpler, and once the operator has pushed the set of wells 11 into the recess between the jaws 43, 243 it is held securely. Once the reagents have been dispensed and the above-described mixing steps completed, the controller 18 operates the upright linear actuator 89 to expose the mixture to the magnetic field, performing the separation that pulls the magnetic microspheres toward the closed ends 21.

The immunoassay device 10, 210 is programmed to perform (in a full auto-analyzer mode, and after the set of wells 11 are secure in the machine) the consecutive steps, a) adding one reagent, b) reciprocating the wells for mixing, c) adding the other reagent, d) reciprocating the wells for mixing, e) magnetic separation and f) fluoroscopy, as these steps are described above. For added versatility, the machine is provided with additional user-selectable operating programmes for performing different subsets of these steps a) to f). For instance, for a small batch of tests the user may manually add the reagents, before selecting a programme that performs only the above steps d) to f). Alternatively, only the magnetic separation and fluoroscopy steps e) and f) may be performed by a different programme, as might follow manual addition of the reagents and mixing. Another programme may provide only for the steps d) and e), perhaps when a visual check of a colour change would suffice, so fluoroscopy is not required. Yet another programme may allow the device the device to be used as a benchtop mixer, only reciprocating the wells for mixing.

In a preferred embodiment of the invention to detect antigen (e.g., osteopontin or OPN) from an unknown sample, the following protocol is adopted based on the current configurations of the immunoassay device 10, 210:
1. Manually pipette 10 μl-50 μl, preferably 30 μl, of the unknown sample to a reaction well; fill the other five wells if necessary and load the whole set of reaction wells to the immunoassay device 10, 210.
2. Use the immunoassay device 10, 210 to dispense 10 μl-50 μl, preferably 20 μl, of the first reagent comprising of magnetic or fluorescent particles, preferably magnetic particles, coated with antibodies to OPN, to the well. Preferably, for detecting large antigens (>1000 daltons) that contain multi-epitopes, the coating antibodies are comprised of a mixture of monoclonal antibodies with different sub-specificities for the antigen, or comprised of polyclonal antibodies.
3. Use the immunoassay device 10, 210 to mix for 1-5 min, preferably 2 min, and immediately dispense 20 μl-100 μl, preferably 50 μl, of the second reagent comprising of fluorescent or magnetic particles, preferably fluorescent particles, coated with the OPN antigen, and mix for 1-5 min, preferably, 2 min.
4. Use the immunoassay device 10, 210 to sediment the magnetic particles and use the reader to read the results.

For samples that contain only small amounts of antigen, the following modification is used to increase the sensitivity of the test:
1. Manually pipette a bigger volume (e.g., 60 μl-200 μl) of the unknown sample to the reaction well and manually deliver 20 μl of the first reagent to the well. NB. This first reagent must be comprised of the magnetic particles and not the fluorescent particles.
2. Use the machine to mix for 1-5 min, preferably, 4 min, and then sediment the magnetic particles.
3. Manually remove the supernatant leaving 50 μl behind; gently and thoroughly re-suspend the sediment in the solution using a pipette.
4. Place the set of wells in the machine and use the machine to dispense 20 μl-80 μl, preferably 50 μl, of the second reagent and complete the rest of the machine operation as before.

In another preferred embodiment of the invention but this time to detect antibodies (e.g., anti-Salmonella LPS antibodies) from an unknown sample, the following protocol is adopted based on the current configuration of the fluorescence reader and the reaction wells:
1. Manually pipette 10 μl-50 μl, preferably 30 μl, of the unknown sample to a reaction well; fill the other five wells if necessary and load the whole set of reaction wells to the immunoassay device 10, 210.
2. Use the immunoassay device 10, 210 to dispense 10 μl-50 μl, preferably 20 μl, of the first reagent comprising of magnetic or fluorescent particles, preferably magnetic particles, coated with the LPS antigen, to the well.
3. Use the immunoassay device 10, 210 to mix for 1-5 min, preferably 4 min, and immediately dispense 20 μl-100 μl, preferably 50 μl, of the second reagent comprising of fluorescent or magnetic particles, preferably fluorescent particles, coated with anti-LPS antibodies, and mix for 1-5 min, preferably, 2 min.
4. Use the machine to sediment the magnetic particles and use the reader to read the results.

For samples that contain only small amounts of antibodies, the following modification is used to increase the sensitivity of the test:
1. Manually pipette a bigger volume (e.g., 60 μl-200 μl) of the unknown sample to the reaction well and manually deliver 20 μl of the first reagent to the well. NB. This first reagent must be comprised of the magnetic particles and not the fluorescent particles.
2. Use the immunoassay device 10, 210 to mix for 1-5 min, preferably, 4 min, and then sediment the magnetic particles.
3. Manually remove the supernatant leaving 50 μl behind; gently and thoroughly re-suspend the sediment in the solution using a pipette.
4. Place the set of wells in the immunoassay device 10, 210 and use the machine to dispense 20 μl-80 μl, preferably 50 μl, of the second reagent and complete the rest of the machine operation as before.

Mixing periods during which the transport is operated to reciprocate the set of reaction wells along the path for mixing are critical to the immunoassay method. There are two separate mixing periods. Table 4 below illustrates, for an analyte comprising Covid-NP antigen, the influence of the first mixing period on the sensitivity of the quantitative measurement, while keeping the second mixing period constant. Table 4 also illustrates the greater sensitivity that could be achieved using a mixture of monoclonal antibodies of different sub-specificities than a single monoclonal antibody.

TABLE 4
Importance of the first mixing step and the use of multiple antibodies
to the assay sensitivity

| | Mixing period | | |
|---|---|---|---|
| | 0 min | 2 min | 4 min |
| Magnetic particles* coated with: | Results in rfu (higher score, higher sensitivity) | | |
| mAb1 (monoclonal antibody | 0 rfu | 37 rfu | 180 rfu |
| with sub-specificity type 1) | | | |
| mAb2 (monoclonal antibody | 82 rfu | 170 rfu | 393 rfu |
| with sub-specificity type 2) | | | |
| mAb1 + mAb2 ** | 371 rfu | 1,031 rfu | 1,108 rfu |

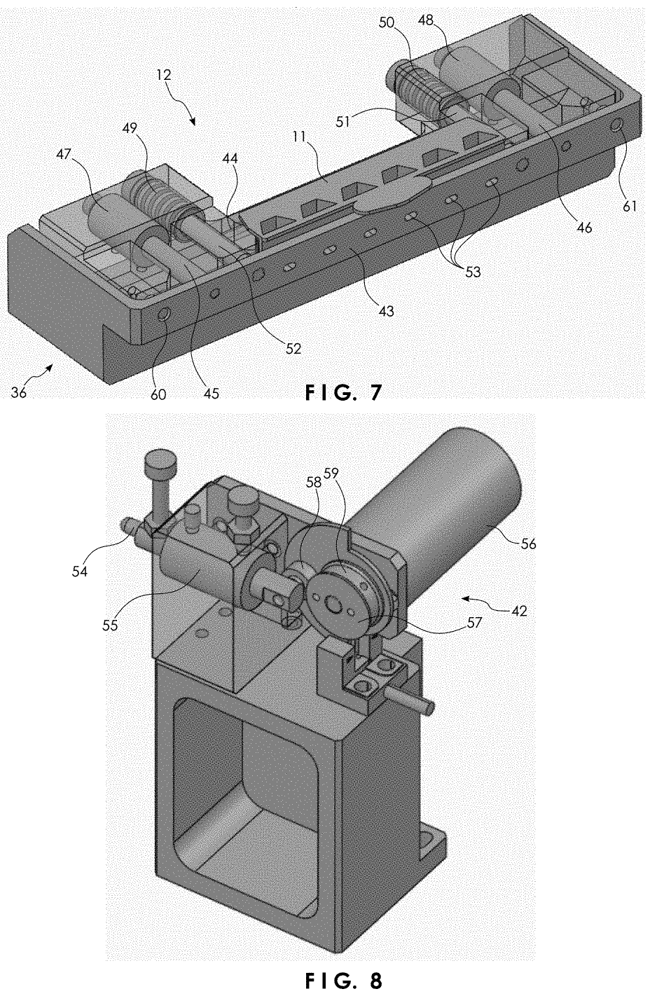

METHOD: Analyte (Covid-NP antigen, 30 μl, same conc. used throughout) mixed
with first reagent (mAb-coupled magnetic particles specific for Covid-NP, 20 μl) for
0, 2, or 4 min; second reagent (fluorescent particles coupled with Covid-NP antigen,
70 μl) then added and mixed for 2 min. Magnetization applied and the results read
by fluorescence reader in relative fluorescence units (rfu). (Mixing, magnetization
and fluorescence reading all done by device depicted in FIGS. 2-9.)
RATIONALE FOR USING ONE OR TWO MIXING STEPS: The TUBEX ® test is
based on the ability of an analyte to inhibit the binding between a pair of reagent
particles. This can be carried out in two ways: (a) incubating the analyte with both
partners of the binding pair at the same time (one-step mixing); (b) incubating the
analyte with just one of the binding partners first (first mixing) and then incubating
altogether with the other binding partner (second mixing). The first option saves
time. It is not clear whether the second option yields better sensitivity as there is no
formal proof.
RATIONALE FOR EXPERIMENTING WITH TWO MONOCLONAL ANTIBODIES:
This is based on the applicant's past experience with the Salmonella O9 antigen.
They found that this antigen could be readily detected in the TUBEX ® test (see
FIG. 12b) as a result of the multi-valency of the antigen i.e. the O9 antigen exists
as multiple, repeating epitopes in the bigger antigen complex called
lipopolysaccharide (LPS). This is different from the many epitopes found in other
antigens, including Covid-NP, which are monovalent i.e. only one copy of the
epitope exists in the whole antigen complex, detectable by only one sub-specificity
of mAb. This makes detection difficult because the chance meeting between
antigen and antibody is slim and the resultant single-point binding could be weak.
The applicant therefore reasoned that the problem might be alleviated using
multiple mAbs of different sub-specificities in order to achieve multi-point
engagement.
*Same no. of particles used in all cases
**Half-concentrations of mAb1 and mAb2 used compared to individual mAbs above

Aspects of the present invention have been described by way of example only and it should be appreciated that modifications and additions may be made thereto without departing from the scope thereof.

1. An immunoassay device for use in performing rapid immunodiagnostic tests to quantitatively measure an amount of an analyte in a fluid sample, comprising:
a set of reaction wells;
a transport which moves the set of reaction wells along a path;
first and second reagent holders disposed alongside the path for holding respective reagents;
a dispenser configured for withdrawing reagent from the reagent holders and dispensing the reagent into ones of the reaction wells, wherein the reagents comprise: a labelled reagent including one of a binding pair coupled with a label, and a magnetic reagent including a magnetic particle coupled with the other of the binding pair;
a magnet disposed alongside the path for applying a magnetic field in a magnetization direction to the contents of the set of reaction wells such that bound pairs are thereby separated;
a photosensitive detector configured to quantitatively measure the amount of analyte; and
a controller operable to coordinate movement of the transport with operation of the dispenser for dispensing of the reagents and operating the transport to reciprocate the set of reaction wells along the path for mixing the fluid sample with the reagents.

2. The immunoassay device of claim 1, wherein the label comprises one of a fluorescent label, a chemilumiscent label and a dye.

3. The immunoassay device of claim 1, wherein the labelled reagent further comprises a non-magnetic particle, the non-magnetic particle coupled to the label and to the one of the binding pair.

4. The immunoassay device of claim 1, wherein the set of reaction wells comprises like reaction wells arrayed in a longitudinal direction, each well extending down from an opening to a closed end, each well having substantially the same cross section throughout its height.

5. The immunoassay device of claim 4, wherein the reaction wells are generally trapezium-shaped in cross section, with a pair of transversely opposing outer walls forming bases of the trapezium, the transversely opposing outer walls comprising at least opposing windows of transparent material, the outer walls aligned substantially parallel to the path.

6. The immunoassay device of claim 5, wherein the trapezium is an acute trapezium, the opposing walls are aligned in the longitudinal direction of the array and the reaction wells of the set are integrally formed.

7. The immunoassay device of claim 4, wherein the openings are arrayed in a top flange that is generally flat and elongated in the longitudinal direction and serves to integrally connect tops of the reaction wells.

8. The immunoassay device of claim 1, wherein the path is linear and parallel to the longitudinal direction.

9. The immunoassay device of claim 1, wherein the dispenser comprises first and second pipette modules, each pipette module dispensing reagent rom a respective one of the reagent holders.

10. The immunoassay device of claim 9, wherein the controller operates each pipette module to draw in a first volume and subsequently dispenses a fraction of the first volume into each of the reaction wells.

11. The immunoassay device of claim 9, wherein the controller operates each pipette module to alternately draw in and expel one or each of the reagents for mixing the reagent prior to dispensing the reagent.

12. The immunoassay device of claim 1, further comprising opposing jaws mounted on the transport, resilient means for urging one of the jaws toward the other from a released position to an engaged position in which the set of reaction wells is clamped between the jaws.

13. The immunoassay device of claim 12, wherein the jaws are elongated in the longitudinal direction and clampingly engage at least one of the outer walls, at least one of the jaws having a respective array of windows, such that each window can be disposed in registration with one of the outer walls of each reaction well.

14. The immunoassay device of claim 12, wherein the transport is moveable along the path under control of the controller to a release station, wherein the release station comprises at least one actuator that is moveable under control of the controller to abut and move the at least one of the jaws from its engaged position to its released position.

15. The immunoassay device of claim 12, wherein a pair of parallel linear guides on the transport support longitudinally opposing ends of the one of the jaws for transverse movement and the at least one actuator comprises a corresponding pair of actuators, each actuator moveable simultaneously under control of the controller to abut and move the at least one of the jaws from its engaged position to its released position.

16. The immunoassay device of claim 15, wherein each actuator comprises a shaft mounted in a linear bushing for movement between a retracted position and an extended position for abutting the one of the jaws, each actuator driven by a rotary motor that turns a cam, wherein a cam follower engaged with the cam is connected to the shaft such that a lobe of the cam displaces the cam follower and the shaft to the extended position.

17. An immunoassay method for quantitatively measuring an amount of an analyte in a fluid sample, comprising:
providing a transport that moves along a path;
providing a set of reaction wells holding a fluid sample;
mounting the set of reaction wells to the transport;
providing in respective ones of two reagent holders a) a labelled reagent including one of a binding pair coupled with a label, and b) a magnetic reagent including a magnetic particle coupled with the other of the binding pair;
operating a dispenser for withdrawing reagent from one of the two reagent holders;
coordinating movement of the transport with operation of the dispenser to dispense the withdrawn reagent into one of the reaction wells, and,
operating the transport to reciprocate the set of reaction wells along the path for mixing the fluid sample with the reagents,
before subsequently applying a magnetic field in a magnetization direction to the contents of the set of reaction wells such that bound pairs are thereby separated, and
operating a photosensitive detector to quantitatively determine the amount of analyte.

18. The method of claim 17 performed using an immunoassay device comprising:
a set of reaction wells,
a transport which moves the set of reaction wells along a path,
first and second reagent holders disposed alongside the path for holding respective reagents,
a dispenser configured for withdrawing reagent from the reagent holders and dispensing the reagent into ones of the reaction wells, wherein the reagents comprise: a labelled reagent including one of a binding pair coupled with a label, and a magnetic reagent including a magnetic particle coupled with the other of the binding pair,
a magnet disposed alongside the path for applying a magnetic field in a magnetization direction to the contents of the set of reaction wells such that bound pairs are thereby separated,
a photosensitive detector configured to quantitatively measure the amount of analyte, and
a controller operable to coordinate movement of the transport with operation of the dispenser for dispensing of the reagents and operating the transport to reciprocate the set of reaction wells along the path for mixing the fluid sample with the reagents, the method further including:
dispensing one of the magnetic reagent and labelled reagent into the reaction wells by the dispenser of the immunoassay device; and
dispensing the other of the magnetic reagent and labelled reagent into the reaction wells outside of the immunoassay device.

19. The method of claim 17, comprising first and second mixing periods during which the transport is operated to reciprocate the set of reaction wells along the path for mixing, the first mixing period following the dispensing of one of the magnetic reagent and labelled reagent, the second mixing period following the dispensing of the other of the magnetic reagent and labelled reagent.

20. The method of claim 17, wherein the label comprises one of a fluorescent label, a chemilumiscent label and a dye.

21. The method of claim 17, wherein the labelled reagent further comprises a first particle, the first particle coupled to the label and to the one of the binding pair.

22. The method of claim 19, wherein, for the detection of an antigen, the binding pair comprises an antigen and antibody pair and the first particle is coupled with the antibody, and the magnetic particle is coupled with the antigen, and the transport first reciprocates the reaction wells to mix the analyte and one of the reagents of the first reagent pair, before dispensing of the other of the reagents of the first reagent pair.

23. The method of claim 19, wherein, for the detection of an antibody, the binding pair comprises an antigen and antibody pair and the first particle is coupled with the antigen, and the magnetic particle is coupled with the antibody, and the transport first reciprocates the reaction wells to mix the analyte and one of the reagents of the first reagent pair, before dispensing of the other of the reagents of the first reagent pair.

24. The method of claim 17, wherein the photosensitive detector is used to measure one of florescence, chemiluminescence and colour.

25. The method of claim 22, wherein the results derived from the photosensitive detector are output in analog form, with an indicator highlighting where a result lies on a scale.

26. The method of claim 17, further comprising adding to the fluid sample a second reagent pair, the second reagent pair having a specificity and label differing from those of the first reagent pair, and
operating the photosensitive detector to quantitatively determine the amount of a second analyte.

27. The method of claim 17, wherein the antibody used for coupling the first particle is a mixture of monoclonal antibodies with different sub-specificities for the corresponding antigen.

28. The method of claim 17, wherein the antibody used for coupling the magnetic particle is a mixture of monoclonal antibodies with different sub-specificities for the corresponding antigen.

29. The method of claim 17, wherein the dispenser comprises first and second pipette modules, and wherein operating the dispenser comprises withdrawing reagent from a respective one of the two reagent holders using a respective one of the first and second pipette modules.

30. The method of claim 17, wherein the reaction wells are generally trapezium-shaped in cross section, with a pair of transversely opposing outer walls forming bases of the trapezium, the transversely opposing outer walls comprising at least opposing windows of transparent material, the outer walls aligned substantially parallel to the path.

31. An immunoassay method for quantitatively measuring an amount of a first analyte in a fluid sample, the sample further comprising a first reagent pair comprising a) a labelled reagent including one of a binding pair coupled with a label, and b) a magnetic reagent including a magnetic particle coupled with the other of the binding pair, the method comprising:
providing a transport that moves along a path;
providing a set of reaction wells holding a fluid sample
mounting the set of reaction wells to the transport;
operating the transport to reciprocate the set of reaction wells along the path for mixing the fluid sample with the reagents,
before subsequently applying a magnetic field in a magnetization direction to the contents of the set of reaction wells such that bound are thereby separated, and
operating a photosensitive detector to quantitatively determine the amount of the first analyte.

32. The method of claim 31 wherein the reaction wells are generally trapezium-shaped in cross section, with a pair of transversely opposing outer walls forming bases of the trapezium, the transversely opposing outer walls comprising at least opposing windows of transparent material, the outer walls aligned substantially parallel to the path.
